(19) United States
(12) Patent Application Publication
(10) Pub. No.: US 2023/0011521 A1
(43) Pub. Date: Jan. 12, 2023

(54) SYSTEM AND METHOD FOR ADJUSTING HOSPITAL UNIT CAPACITY BASED ON PATIENT-SPECIFIC VARIABLES

(71) Applicant: KONINKLIJKE PHILIPS N.V., Eindhoven (NL)

(72) Inventors: Syamanthaka Balakrishnan, Bangalore (IN); David Paul Noren, Cambridge, MA (US); Gregory Boverman, Cambridge, MA (US); Vishnu Raj, Bangalore (IN); Lasith Adhikari, Cambridge, MA (US); Eran Simhon, Cambridge, MA (US)

(21) Appl. No.: 17/855,958

(22) Filed: Jul. 1, 2022

Related U.S. Application Data
(60) Provisional application No. 63/218,538, filed on Jul. 6, 2021.

(51) Int. Cl. G06N 3/02; G06N 20/00

(52) U.S. Cl. CPC G06N 3/02; G06N 20/00

(57) ABSTRACT

A method for performing a demand analysis for a hospital, including: (i) receiving hospital capacity information; (ii) receiving hospital data, the hospital data comprising information on patient admissions, patient discharges, and patient transfers for a previous period of time for each of a plurality of patient types; (iii) adapting parameters of a machine learning algorithm based on the hospital data; (iv) receiving clinical information about patients currently admitted in the hospital; and (v) determining, based on output from the adapted machine learning algorithm and using the current clinical information and the hospital capacity information, a predicted patient flow for the hospital in real-time. The method further includes displaying, to at least one user in real-time, the predicted patient flow for the ward and at least one suggested rearrangement of resources within the hospital.

### FIELD OF THE DISCLOSURE

The present disclosure is directed generally to methods and systems for predicting patient transitions and/or demand and reallocating beds and/or other resources to optimize the flow of patients within a hospital and balancing hospital resources, enabling informed clinical decisions.

### BACKGROUND

Hospitals have a certain capacity based on the specialization or category of medical services offered. The hospital determines the capacity of each ward or unit within the hospital. However, since the patient in-flow is dynamic, there may be an administrative need to modify or adjust the capacities of wards or units, so that existing patients can be well accommodated for and given appropriate care. There may also be a need to modify or adjust the capacities of wards or units so that new patients don't need to be turned away.

Currently, hospital staff often meet in daily “capacity huddles” with information displayed on whiteboards and communication taking place primarily via email or telephone calls. Many large institutions often find themselves in capacity emergencies. For example, a sudden surge in a number of patients arriving at a hospital can make the hospital turn away patients or redirect them to other hospitals. Such a sudden surge can cause the hospital to lose business and/or delay the hospital's patients from receiving the care they need. A clinical turn of events can indicate an increased need of resources of specific wards for in-patients. However, planning resources and staff and administrative control can be challenging for hospitals due to the variety of uncertainties, such as, in patient arrivals, varying patient types, administrative, and structural changes.

The general strategy for improving resource utilization is through patient flow modeling. Such modeling helps in understanding potential bottlenecks in staffing and resource utilization. However, current models do not automatically adapt over time and do not consider patient type-specific data in calibrating model parameters. Predictive models or hospital simulations solely trained by historical admission, discharge, and transfer data tend to lack in accuracy and may prove to be ineffective when such historical data is not adequately available.

### SUMMARY OF THE DISCLOSURE

There is a need for methods and systems that predict increases in demand for hospital resources with sufficient advance notice without investing the time and effort of manual and on-the-spot resource rearrangements. Such methods and systems can help a hospital make informed decisions to modify the capacities of the hospital or sub-wards or units of the hospital temporarily, for example, to handle a sudden surge of new patients or expand wards with higher resources, such as, in intensive care units or isolation units, to match the stay and transitions of current in-patients. There is also a need for methods and systems that provide a multitude of probable necessary demand adjustments or solutions, with minimal negative effects, and demonstrate the gamut of savings for each solution.

The present disclosure is directed at inventive methods and systems for predicting a patient flow for a hospital in real-time using modeling techniques that automatically adapt over time and take into consideration patient type-specific data in calibrating model parameters. Various embodiments and implementations herein are directed to a system or method that comprises patient flow modeling to assist in modifying a capacity of a hospital or a sub-ward or unit of a hospital. The system or method receives hospital capacity information and hospital data about the hospital, wherein the hospital data includes information on patient admissions, patient discharges, and patient transfers for a previous period of time for a plurality of patient types. A machine learning algorithm is adapted based on the hospital data. The system or method receives clinical information about a plurality of patients currently admitted to the hospital and determines, based on output from the adapted machine learning algorithm and using the clinical information about the patients currently admitted to the hospital and the hospital capacity information, a predicted patient flow for the hospital in real-time. The predicted patient flow can include inflow, occupancy, and outflow for a period of time, such as, 12-24 hours in the future for the hospital or a sub-ward or unit of the hospital. The system or method displays the predicted patient flow to a user on a display and one or more suggested rearrangements of resources. The displayed data assists the user in modifying a capacity of the hospital or a sub-ward or unit of the hospital.

Generally, in one aspect, a method for performing, using a patient flow system, a demand analysis for a hospital to optimize a flow of patients with the hospital is provided. The method includes receiving or accessing by the patient flow system, hospital capacity information about the hospital; receiving or accessing, by the patient flow system, hospital data about the hospital, wherein the hospital data comprises information on patient admissions, patient discharges, and patient transfers for a previous period of time for each of a plurality of patient types; adapting, by a processor of the patient flow system, parameters of a machine learning algorithm based on the hospital data; receiving or accessing, by the patient flow system, clinical information about a plurality of patients currently admitted to the hospital; determining, by the processor of the patient flow system based on output from the adapted machine learning algorithm and using the clinical information about the plurality of patients currently admitted in the hospital, and the hospital capacity information, a predicted patient flow for the hospital in real-time; and displaying, on a user interface, the predicted patient flow for the hospital in real-time to at least one user, wherein the predicted patient flow is configured to assist the at least one user in modifying a capacity of the hospital.

According to an embodiment, the method further includes displaying, on the user interface, to the at least one user a plurality of suggested rearrangements of resources within the hospital based on the generated predicted patient flow.

According to an embodiment, the method further includes receiving a selection, by the at least one user using the user interface, of a first suggested rearrangement of resources of the plurality of suggested rearrangements of resources; and displaying, on the user interface, how the selected first suggested rearrangement of resources would affect the hospital for at least a few hours in the future from the step of receiving the selection of the first suggested rearrangement of resources.

According to an embodiment, the method further includes displaying, on the user interface how the selected first suggested rearrangement of resources would affect the hospital for more than 24 hours in the future from the step of receiving the selection of the first suggested rearrangement of resources.

According to an embodiment, the method further includes receiving a selection, by the at least one user using the user interface, a second suggested rearrangement of resources of the plurality of suggested rearrangements of resources; and displaying, on the user interface, how the selected second suggested rearrangement of resources would affect the hospital for at least a few hours in the future from the step of receiving the selection of the second suggested rearrangement of resources.

According to an embodiment, the method further includes adopting at least one suggested rearrangement of resources of the plurality of suggested rearrangement of resources after receiving user input from the at least one user at the user interface; modifying the hospital capacity information based on the adopted at least one suggested rearrangement of resources; and incorporating at least one change to the resources in the hospital.

According to an embodiment, the method further includes modifying the hospital capacity information for a fixed duration, after which, automatically resetting the hospital capacity information to an original or previous configuration.

According to an embodiment, the method further includes providing feedback to a simulation engine by inputting the adopted suggested rearrangement into the simulation engine thereby updating a basis for a subsequent simulation.

According to another aspect, a patient flow system configured to perform a demand analysis for a hospital to optimize a flow of patients within the hospital is provided. The system includes hospital capacity information about the hospital; hospital data about the hospital, wherein the hospital data comprises information on patient admissions, patient discharges, and patient transfers for a previous period of time for each of a plurality of patient types; clinical information about a plurality of patients currently admitted in the hospital; a machine learning algorithm configured to be adapted based on the hospital data about the hospital; a processor configured to: (i) adapt parameters of the machine learning algorithm based on the hospital data; and (ii) generate a predicted patient flow for the hospital in real-time based on: (1) output from the adapted machine learning algorithm, and (2) the clinical information about the plurality of patients currently admitted in the hospital, and (3) the hospital capacity information; and a user interface communicably coupled with the processor, the user interface configured to display the predicted patient flow for the hospital in real-time for at least one user, wherein the predicted patient flow is configured to assist the at least one user in modifying a capacity of the hospital.

According to an embodiment, the user interface is further configured to display, to the at least one user, a plurality of suggested rearrangements of resources within the hospital based on the generated predicted patient flow.

According to an embodiment, the user interface is further configured to (i) receive user input from the at least one user selecting a first suggested rearrangement of resources of the plurality of suggested rearrangements of resources; and (ii) display, to the at least one user, how the selected first suggested rearrangement would affect the hospital for at least a few hours in the future after receiving the user input.

According to an embodiment, the user interface is further configured to (i) receive user input from the at least one user selecting a second suggested rearrangement of resources of the plurality of suggested rearrangements of resources; and (ii) display, to the at least one user, how the selected second suggested rearrangement of resources would affect the hospital for at least a few hours in the future after receiving the user input.

According to an embodiment, the processor is further configured to (i) adopt at least one suggested rearrangement of resources of the plurality after receiving user input from the at least one user at the user interface; and (ii) modify the hospital capacity information based on the adopted at least one suggested rearrangement of resources.

According to an embodiment, the hospital capacity information is modified for a fixed duration, after which the hospital capacity information is automatically reset to an original or previous configuration.

According to an embodiment, the processor is further configured to provide feedback to a simulation engine by inputting the adopted suggested rearrangement into the simulation engine thereby updating a basis for a subsequent simulation.

In various implementations, a processor or controller may be associated with one or more storage media (generically referred to herein as “memory,” e.g., volatile, and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a schematic representation of a patient flow system, in accordance with an embodiment.

FIG. 2 is a schematic representation of a patient flow system, in accordance with an embodiment.

FIG. 3 is a schematic representation of a flow of data through adaptive parameter modeling and simulation, in accordance with an embodiment.

FIG. 4 is a flowchart of a method for performing a demand analysis of a ward or unit of a hospital using a patient flow system, in accordance with an embodiment.

FIG. 5 is an example schematic graphical representation of a display of a plurality of suggested rearrangements of resources, in accordance with an embodiment.

FIG. 6 is an example process for performing simulation feedback, in accordance with an embodiment.

FIG. 7 is a flowchart of a method for performing a demand analysis using a patient flow system, in accordance with an embodiment.

### DETAILED DESCRIPTION OF EMBODIMENTS

The present disclosure describes various embodiments of a system and method for predicting patient arrivals, transitions, and/or demand and reallocating beds and/or other resources to optimize a flow of patients within a hospital and balance hospital resources. Applicant has recognized and appreciated that it would be beneficial to leverage hospital data comprising previous patient admissions, previous patient discharges, and previous patient transfers and clinical information about patients currently admitted to the hospital (e.g., patient vitals, laboratory procedures, medications, clinician observations and co-morbidities, pre-existing medical conditions, symptoms, reasons for visits and medication information) to accurately predict in advance, and in real-time, one or more estimated resource adjustments for a hospital or a ward or unit of the hospital based on a predicted demand for resources in the hospital or each ward, within the limits of the overall hospital capacity. Example methods receive or access hospital capacity information about a hospital, previous admission, discharge, and transfer data for a plurality of patient types, and a machine learning algorithm uses the received or accessed admission, discharge, and transfer data to continuously or periodically update or adapt modeling parameters. Example methods further receive or access clinical information about patients currently admitted to the hospital. The method further determines a predicted patient flow for the hospital in real-time based on output from the adapted machine learning algorithm and using the clinical information about the patients currently admitted to the hospital and the hospital capacity information. The predicted patient flow can include a recommendation to make an adjustment to the hospital capacities for the optimal cost, resource utilization, patient comfort, and/or staff satisfaction. The method further includes displaying the predicted patient flow for the hospital in real-time for at least one user (e.g., hospital management personnel) wherein the predicted patient flow is configured to assist the at least one user in modifying a capacity of the hospital or a sub-ward or unit of the hospital.

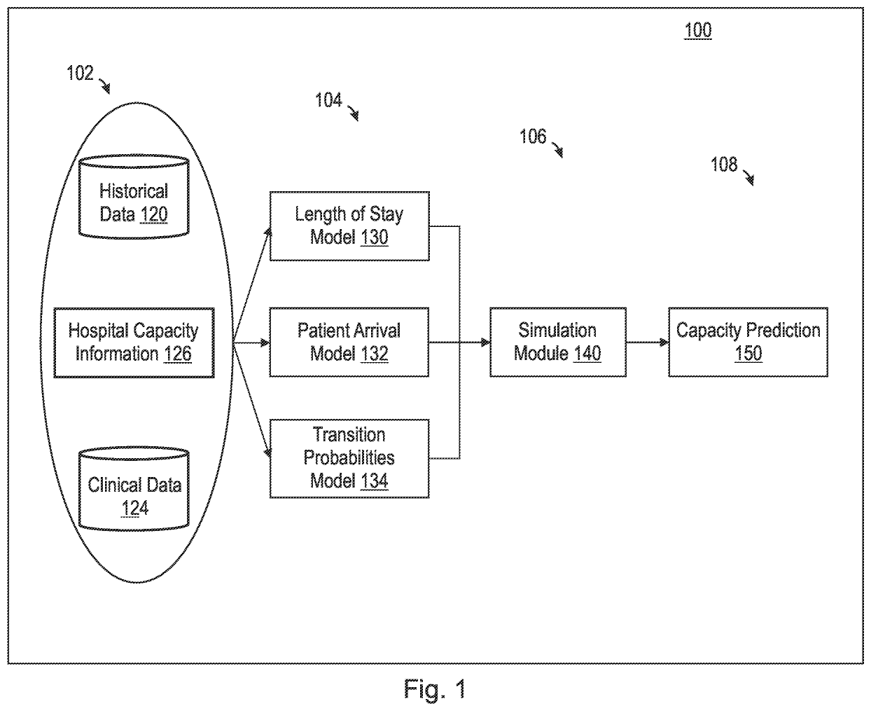

Referring to FIG. 1, in one embodiment, is a schematic representation of a patient flow system 100. Patient flow system 100 may be any of the systems described or otherwise envisioned herein, and may comprise any of the components described or otherwise envisioned herein.

According to an embodiment, system 100 includes input data 102, various models 104, simulation engine 106, and output 108. Input data 102 can be considered as a data layer, models 104 and simulation engine 106 can be considering a modeling layer, and output 108 can be considered an access layer. As further described herein, the modeling layer comprises various models and analytics and the access layer comprises a user interface-based tool to assist a clinical decision maker in making informed decisions. Input data 102, or the data layer, comprises a data adapter that is configured to read all required electronic medical record data including clinical notes (e.g., encounter, encounter location history, admit/final diagnosis, co-morbidities, clinical events, past hospital and Emergency Department (ED) visit data, and physiologic measurements, etc.) in real-time. In embodiments, the electronical medical records are in fast healthcare interoperability resources (FHIR) format developed by the Health Level Seven International (HL7) healthcare standards organization. The adapter of the data layer can be configured to perform variable generation and pre-processing on the electronic medical record data. The adapter of the data layer can be programmed to maintain a two year window of data such that as the new data comes in, old data is removed. Of course, any time period window is contemplated. As shown in FIG. 1, the electronic medical record data comprises any of hospital data 120, clinical data 122, and hospital capacity information 126. Any of the electronic medical record data can be retrieved from a database that is part of a hospital data repository/IT system. The adapter of the data layer can be configured to put the data into a common format in embodiments.

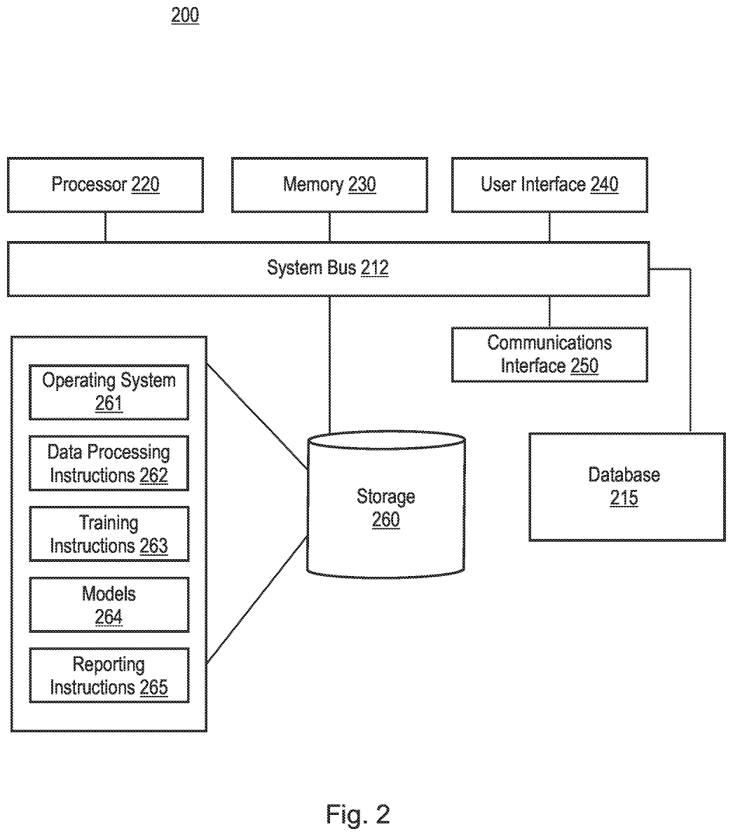

Referring to FIG. 2, in one embodiment, is a schematic representation of a patient flow system 200. System 200 comprises one or more of a processor 220, memory 230, user interface 240, communications interface 250, and storage 260, interconnected by one or more system buses 212. It will be understood that FIG. 2 constitutes, in some respects, an abstraction and that the actual organization of the components of the system 200 may be different and more complex than illustrated.

According to an embodiment, system 200 comprises a processor 520 capable of executing instructions stored in memory 230 or storage 260 or otherwise processing data to, for example, perform one or more steps of the methods described herein. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may comprise or be in remote or local communication with a database or data source 215. Database 215 may be a single database or data source or multiple databases or data sources. Database 215 may comprise the input data which may be used to train the models, as described and/or envisioned herein. The input data may also be the data for a specific patient or patients in a specific ward or unit of a hospital for which the demand analysis is being performed. As an example, the input data can include detailed information on patient demographics such as age, gender, and more; diagnosis or medical condition such as cardiac disease, psychological disorders, chronic obstructive pulmonary disease, and more; physiological vital signs such as heart rate, blood pressure, respiratory rate, oxygen saturation, and more; and/or current and/or past data. Many other types of input data are possible. Accordingly, database 215 may be an electronic medical record database or any other type of database.

According to an embodiment, storage 260 of system 200 may store one or more algorithms, models, and/or instructions to carry out one or more functions or steps of the methods described or otherwise envisioned herein. For example, processor 220 may comprise one or more of data processing instructions 262, training instructions 263, models 264, and/or reporting instructions 265.

According to an embodiment, data processing instructions 262 direct the system to retrieve and process input data which is used to either: (i) train or adapt the models 264 using the training instructions 263, or (ii) to perform a demand analysis for a hospital or a sub-ward or unit of a hospital using the models 264. The data processing instructions 262 direct the system to, for example, receive or retrieve input data or medical data to be used by the system as needed, such as from database 215 among many other possible sources. As described above, the input data can comprise a wide variety of input types from a wide variety of sources.

According to an embodiment, the data processing instructions 262 also direct the system to process the input data to generate a plurality of features related to medical information for a plurality of patients, which are used to train models 264. This can be accomplished by a variety of embodiments for feature identification, extraction, and/or processing. The outcome of the feature processing is a set of features related to transfer and/or discharge information and/or outcome for a cohort of previously monitored patients, which thus comprises a training data set that can be utilized to train the models.

According to an embodiment, training instructions 263 direct the system to utilize the processed data to train the models to determine a predicted patient flow for a hospital or a sub-ward or unit of the hospital, wherein the predicted patient flow comprises inflow, occupancy, and/or outflow for a period of time, such as, 12-24 hours in the future. The models can be any suitable model sufficient to utilize the type of input data provided. Thus, the system comprises trained models 264 configured to determine a predicted patient flow for a hospital or a sub-ward or unit of a hospital.

In embodiments, the models 264 include length of stay model 130, patient arrival model 132, transition probabilities model 134, simulation module 140, and capacity estimator model 640 as described herein. The models employ a combination of prior patient transition data of the hospital, clinical information of patient data, and a simulation engine. The data is modeled using machine learning techniques taking into account the seasonality and trends of the data. The modeling techniques adjust parameters in an adaptive way using past and most recent trends and patient patterns in the hospital flow. The length of stay model 130 can be based on an inverted empirical cumulative distribution function (CDF) model. Rather than using a parametric approach for modeling length of stay (LOS), this empirical approach is observed to be robust to fitting past data. Since length of stay (LOS) distributions vary from unit-to-unit within a hospital and also based on patient-type, Applicant has recognized and appreciated that it is crucial to model the LOS distributions individually based on the unit or patient type to preserve their own properties. In embodiments of the adaptive modeling, LOS data per unit or patient type is used for a prior period of time, such as a two year period of time, to generate empirical cumulative distribution functions (CDF). The inverted and interpolated empirical CDFs can be used in real-time to assign length of stay for simulated patients through a suitable uniform random sampling technique.

In embodiments, the length of stay model can be used to condition the empirical CDFs by the hour of admittance for the patients. There can be a clear association of the LOS distributions and the admit hour. For example, a length of stay distribution for patients who arrived at 12 am can be more likely to leave in the same day noon, which is after 12 hours from the admission. A length of stay distribution for patients who arrived at 7 am can be more likely to leave in the next day noon, which is after 29 hours from the admission.

Another advantage of using the inverted empirical CDF model is that since no assumption about the underlying process is exercised in the design, it can be used at any site of deployment without modification and the CDF model still works as expected.

Applicant has further recognized and appreciated that clinical conditions are a major factor influencing a length of stay of a patient. In embodiments, the length of stay model 130 is based on the clinical condition of the patients. Clinical condition information of the patients can be divided into two categories, disease information and physiological data, and combined as described herein or in any other suitable manner. Disease information, or information about one or more diagnosed diseases, plays an important role in predicting an expected length of stay for each patient. The length of stay model 130 can be configured to regress on disease by embedding age, gender, and one or more co-morbidities, for example, to predict an expected length of stay at the time of admittance. The expected length of stay at the time of admittance can be calculated and stored until later combined with the expected residual length of stay as described below.

Physiological or vitals data can be obtained continuously or at selected intervals for each patient. Such data can be used to refine the length of stay prediction based on the disease information. The latest vitals data along with the diagnosis data (e.g., disease characteristics) and time spent by the patient in hospital can be obtained or accessed and a gradient boosting regression method, or any other suitable alternative, can be applied to generate an expected residual length of stay prediction. The calculated expected length of stay at the time of admittance can be combined with the expected residual length of stay, in a linear fashion or any suitable alternative, to generate the expected length of stay and residual length of stay for each patient.

The patient arrival model 132 can be used to adaptively and accurately predict patient arrivals. Since patient arrival patterns vary over time and are difficult to predict, Applicant has recognized and appreciated that it is critical to adjust baseline arrival rates (i.e., monthly arrival rates from the past year) using recent past arrival rates to make a more accurate prediction. The patient arrival model 132 can be based on the Poisson arrival probabilities, where historical data is used to compute hourly arrival rates. The adaptive arrival rate adjustment process can proceed as follows in embodiments. First, hourly arrival rates can be computed on a monthly and day-of-week basis for a period of time prior to the initiation of a given simulation. The period of time can be a twelve month period or any other suitable period of time. Second, a hyper-parameter can be trained for each day-of-week such that it optimizes the weighted adjustment of the baseline arrival rates (i.e., 12 months ago rates) with past four weeks arrival rates. Training of a hyper-parameter, also known as gamma, can ensure that historical and recent trend data is balanced. The gamma can be trained to optimize the weighted adjustments of historical data using the most recent trends, thus achieving a blended model, with higher accuracy in forecasting. The adjusted arrival rates for a given day-of-week “w” and hour “h” can be used to generate arrival counts from the Poisson distribution. The predicted arrival counts can be computed using the following equation λ_{(w,h)}^{adj}=λ₍ₕ₎^{12 months ago}−γ_{(w)}(λ₍ₕ₎^{12 months ago}−λ_{(w,h)}^{last 4 weeks}) wherein λ_{(w,h)} is the average hourly arrivals at (w, h), w is the day of the week [0, 1, 2, . . . , 6], and h is the hour of the day [0, 1, 2, . . . , 23]. The expected patient arrivals/hour is approximately Pois (λ_{(w,h)}^{adj}).

The transition probabilities model 134 is deployed to determine a movement and trajectory of patients within a hospital or within a sub-ward or unit of the hospital. The term transition probability as used herein means a number of patient transitions within a hospital (e.g., from one unit to another) within a certain time period with respect to a total number of outbound transitions. Since transition probabilities do not remain constant over time due to administrative and structural changes in a hospital system for example, accurate predictions cannot be expected when constant transition probabilities are used in a patient simulation. Embodiments utilize transition probabilities model 134 to derive accurate and adaptive transition probability results as described herein. Weekly transition probabilities for patients from an emergency department to other wards or units in a hospital, for example, can be predicted performing short-term transition probability forecasting using recent past transition trends. The predicted transitions are initially based on past patient transition data for a period of time (e.g., 18 months or any suitable period of time) with respect to the current time. The past patient transition data comprises a start unit, an end unit, and a transition time. The past patient transition data can be used to train and test a forecasting model using, e.g., an exponential smoothing method. In embodiments, unit transitions are forecasted by patient-type in order to improve the accuracy of simulated patient trajectories.

The process for adapting transition probabilities can proceed as follows in embodiments. First, information about the encounter location history and admitting diagnosis for patients currently admitted to a hospital are obtained, received, or otherwise accessed. This information may be stored in and/or received from one or more databases. The database may be a local and/or remote database. For example, the patient flow system may comprise a database of training data.

According to an embodiment, the patient flow system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

The information about the encounter location history and admitting diagnosis for patients currently admitted to the hospital is considered with respect to previous patient transition data at the hospital. In other words, the past patient transition data is enriched with clinical information for the patients currently admitted to the hospital. Such clinical information includes vital signs measured continuously, in specific intervals, or micro frequencies, for example, continuous heart rate monitoring or blood glucose monitoring at 6 hour intervals, medication during the hospital stay, any results of laboratorial tests including X-rays, Ultra scans, Mills, etc. In embodiments, apart from immediate vitals information, co-morbidity information and prior medication data, for example, also form part of the clinical information. Second, the information obtained in the first step is pre-processed to generate a clean dataset and weekly transition counts are computed based on the information. The pre-processing can include outlier removal, feature generation, and/or missing data imputation so that the clean dataset represents ideal data elements corresponding to patient type. Next, the weekly transition counts are used to train and test or validate one or more forecasting models using, e.g., an exponential smoothing method or any suitable alternative. The one or more forecasting models can be trained on a portion of the generated clean dataset. In embodiments, the generated clean dataset can be randomly split into a training data cohort and a test data cohort and the training data cohort can be used to train the one or more models. The portion of the clean dataset in the test data cohort can be used to test or validate the one or more models. In embodiments, the forecasting model is Holt's linear forecasting however, other suitable forecasting models can be used as well, including but not limited to vector auto regression, carry forward baseline, among others. Multiple forecasting models can be used in embodiments and suitable performance validation methods can be used to determine a best fit. Next, the one or more forecasting models are trained for forecasting model parameters and transition probabilities are forecasted.

In embodiments, the transition probabilities model 134 uses the weekly transition counts along with the other clinical information to predict or derive a suitable trajectory or transition of the patients currently admitted to the hospital through various wards and/or units. In embodiments, the transition probabilities model 134 uses the weekly transition counts along with the other clinical information to predict a number of patient transitions within a certain time period with respect to a total number of outbound transitions. The forecasted transition probabilities can be based on the identified patient type. In embodiments, the trajectory of the patients through the hospital, and optionally between wards or units of a hospital, is achieved with machine learning based algorithms such as kernel-based methods such as K-nearest neighbor (KNN) classifiers and support vector machine (SVM) classifiers among others. Patient similarity on a multitude of parameters forms an important part of the clustering algorithms.

The models 130, 132, and 134 simulate the trajectory of patients across the hospital or sub-wards or units at an individual level, based on co-morbidities and historic medical conditions and so on, mimicking their probable length of stay, probable number of arrivals to each ward or unit etc. The observable or otherwise measurable traits or characteristics of patients, or phenotyping, helps ensures consistency across arrivals, length of stay, and transition probabilities. Simulation module 140 is configured to receive the outputs from length of stay model 130, patient arrival model 132, and transition probabilities model 134 and make a demand or capacity prediction 150 for the hospital or sub-wards or units of the hospital. In other words, the simulation module 140 takes into account existing patients in the hospital or each ward or unit in the hospital, including their stay and transitions when instantiating the simulation at each time point. Simulation module 140 simulates or mimics the hospital and the patients currently admitted to the hospital digitally within a framework using models 130, 132, and 134 as its pillars. The simulation module 140 reads calibrated parameters from the adaptive parameter modeling module and a list of existing patient details to execute the simulation engine. The framework takes into account the existing patients in the hospital in which the system is deployed and introduces simulated patients at every given time point. The LOS modeling specific properties and ward or unit-related properties (e.g., true capacity, wards to be simulated, overflow policy per ward) can be configured through the hospital specific configurations in the simulation framework. The simulation framework utilizes discrete event simulation and easily interacts and collaborates with multiple units in the simulation network. The term discrete event simulation as used herein refers to the operation of a system as a discrete sequence of events in time. The simulation module 140 generates a digital hospital twin that provides critical information on what is to be expected in terms of patient arrivals, transitions, stays, and exits through the hospital or sub-wards or units at every given time point. Using the demand prediction 150, the simulation module 140 generates a gamut of metrices that can be made available to the user, e.g., hospital management or command center. The metrices can comprise average waiting times, average length of stay, wait times at an emergency department (ED), among others. The simulation module 140 performs in real-time and streams predicted arrivals, occupancy, exits, and capacity of the hospital or each ward or unit of the hospital in advance. In embodiments, the simulation module 140 makes forecasts for 4 hours, 8 hours, 12 hours, 24 hours, and 48 hours ahead, although any suitable intervals are contemplated.

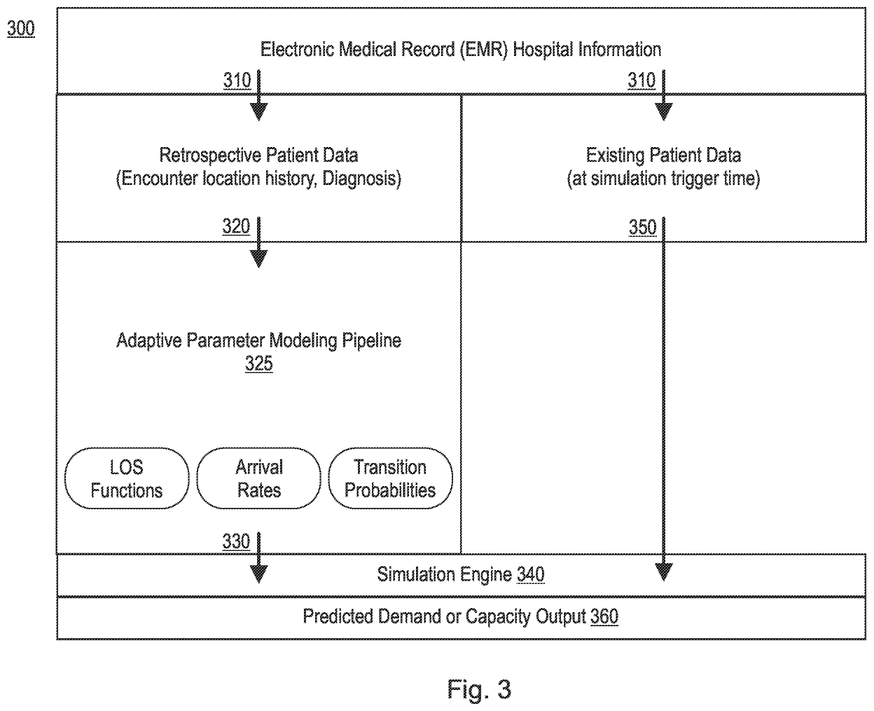

FIG. 3 shows a schematic representation of a flow of data through the adaptive parameter modeling and simulation as described or otherwise contemplated herein. First, electronic medical record hospital information is retrieved, accessed, or otherwise obtained from one or more databases at 310. Then, at 320 retrospective patient data comprising encounter location history and diagnosis information is fed into adaptive parameter modeling pipeline 325. Pipeline 325 comprises the expected LOS and residual LOS from the LOS model 130, predicted arrival counts from patient arrivals model 132, and forecasted patient transition probabilities from model 134. As described herein, at 330 the outputs from the adaptive parameter modeling are input to simulation engine 340, which is akin to simulation module 140. At 350, existing patient data that is provided from the electronic medical record system at 310, is provided to simulation engine 340. At 360, predicted demand or capacity forecasts are output from simulation engine 340. In embodiments, the forecasts are presented for the next 24-48 hours. The forecasts are limited by the available resources as dictated by the hospital capacity information.

As shown in FIG. 3, the simulation engine 340 requires existing patient data when triggered and the modeled parameter inputs: LOS approximation functions, arrival rates; and transition probabilities. In embodiments, the simulation engine 340 is triggered hourly, but any suitable timing is contemplated. In embodiments, the adaptive parameter modeling pipeline 325 is triggered on a weekly basis to update the parameters. Since patient prioritization is highly influenced by the acuity of the patient's condition, the simulation engine 340 enables a user to adopt a deterioration-based hospital or ward or unit queueing. In other words, the simulation engine 340 is configured according to a patient's acuity-based queueing approach.

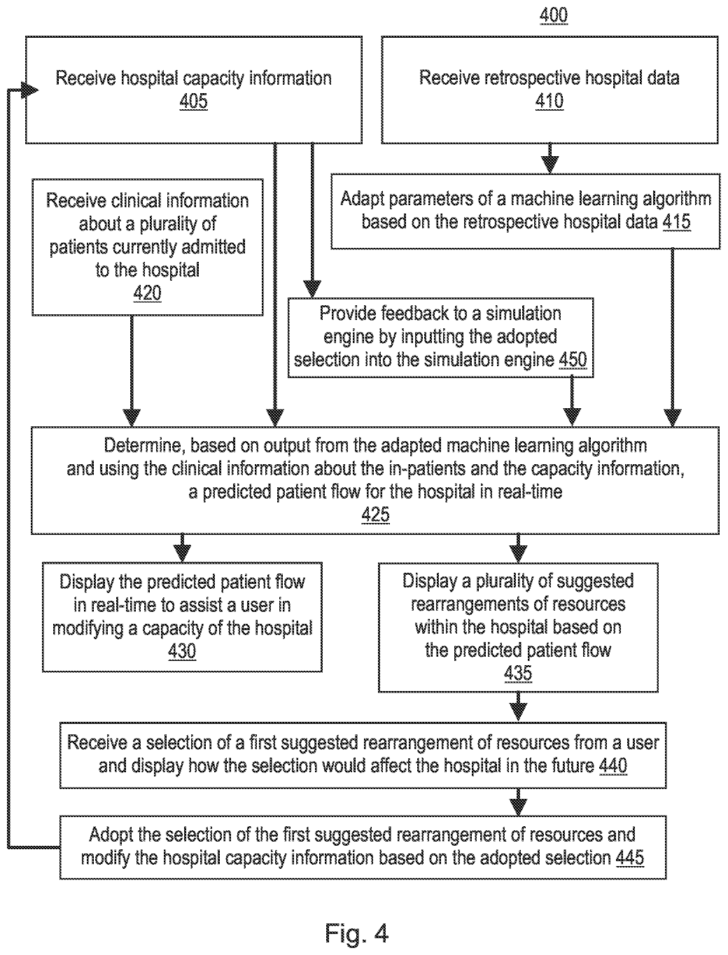

Referring to FIG. 4, in one embodiment, is a flowchart of a method 400 for performing, using a patient flow system, a demand analysis for a hospital to optimize a flow of patients. The patient flow system may be any patient flow system described or otherwise envisioned herein.

As discussed in greater detail herein, the patient flow system uses adaptive historical data to develop models to predict aggregate demand for beds and other resources in a probabilistic manner. In other words, historical data-based simulation is combined with adaptive statistical learning techniques to build the hospital simulation. These models are informed by both recent trends and by specific characteristics of patients who are already in the hospital. The adaptive predictive modeling module provides healthcare institutions actionable information about future demand that allows them to rebalance resources in various ways. The actionable information includes, among others, temporarily reducing or increasing resources (beds/staff) in various locations and also more expeditiously discharging low-acuity patients. The present disclosure focuses on predicting patient arrivals and/or transitions and/or demand and reallocating beds and other resources to optimize flow of patients within the hospital and balancing hospital resources, enabling informed clinical decisions. The emphasis of the present disclosure highlights how the demand or capacity predictions can be used to offer the users suggested optimal adjustments to the hospital or unit capacities. In embodiments, the system or method predicts patient arrivals and trajectories at a hospital based on (i) real-time vitals data, previous medical conditions and/or co-morbidities, or the like, of patients currently in the hospital and (ii) a patient flow at a population level based on past hospital admission/discharge/transfer (ADT) data. In embodiments, the predictions are based on stochastic modeling approaches. In various implementations, uncertainty or confidence levels are provided with each prediction to help decision makers account for the probabilistic nature of the predictions. Various embodiments include a user interface configured to visualize or display these predictions and uncertainty levels on a per-unit or ward basis and various levels of aggregation.

According to an embodiment, the patient flow system may be embodied in whole or in part within a device. For example, the entire patient flow system may be embodied within a single device such as a handheld device, laptop, computer, or other single device. Alternatively, the patient flow system may comprise a user interface that is transportable, such as a handheld device, mobile phone application, computer, or other transportable element that functions as a user interface to receive information at the hospital or sub-ward or unit of the hospital. The device can be configured to communicate the information to another, remote component of the patient flow system for analysis. The result of the patient flow system may then be communicated back to the transportable user interface.

At step 405 of the method, the patient flow system receives input data (e.g., layer 102) comprising hospital capacity information (e.g., 126). At step 410 of the method, the patient flow system receives additional input data (e.g., layer 102) comprising retrospective patient data (e.g., 120, 320). The retrospective patient data can be updated hourly, in any other suitable interval, or continuously. The hospital capacity information can be updated periodically or when it changes. At step 415 of the method, the patient flow system adapts parameters of a machine learning algorithm based on the retrospective hospital data. As described in FIG. 3, retrospective patient data comprising encounter location history and diagnosis information can be fed into an adaptive parameter modeling pipeline (e.g., 325) comprising the expected LOS and residual LOS from the LOS model 130, predicted arrival counts from patient arrivals model 132, and forecasted patient transition probabilities from model 134. At 420 of the method, the patient flow system receives clinical data for existing patients currently admitted to the hospital (e.g., 124, 350). This input data may be stored in and/or received from one or more databases. The database may be a local and/or remote database. At step 425 of the method, the patient flow system determines a predicted patient flow for the hospital in real-time. The predicted patient flow is based on output from the adapted machine learning algorithm and using the clinical information about the in-patients and the capacity information. Step 425 is carried out by simulation module 140 and/or engine 340. The predicted patient flow comprises predicted demand or capacity output 360 as described herein.

Following step 425 of the method, the patient flow system displays the predicted patient flow in real-time to assist a user in modifying a capacity of the hospital at step 430 of the method. At step 435 of the method, the patient flow system displays a plurality of suggested rearrangements of resources within the hospital based on the predicted patient flow. The displayed suggestions can be embodied as a rule-based application that empowers the user to make informed decisions by trying multiple what-if and/or if-this-then-that options and visualizing the various scenarios of staff and resource utilization and effectiveness. These scenarios can be made available to the user up to 72 hours in advance in embodiments. In embodiments, the rule-based application takes into account the total capacity of the hospital and inputs on mixed acuity units of the hospital. The availability of free beds, which can be predicted by the simulation module 140 or engine 340, is an example parameter that can be used by the rule-based application to generate a variety of what-if and/or if-this-then-that options or scenarios. For example, weights and penalties can be applied to the options or scenarios based on user selected changes depending on the resources available, as well as the staffing needs post the user selected change. In an embodiment, a bed in an emergency ward cannot be converted into that of an ICU, as the latter requires availability of resources like ventilators, monitors, and life support machines, for example. In embodiments, the staff-to-patient ratio for each ward or unit forms an integral part of the weightage system. Costs and logistic penalties can be applied to each solution or scenario. Thus, in embodiments, most effective and/or cost efficient suggestions may be generated and displayed by the patient flow system. In embodiments, the patient flow system is configured to handle surge protocols as defined by the hospital, to automatically take effect at defined thresholds. The configurable part of the application can have multiple settings taking into account the needs of a number of hospitals, with options for a fixed bed setting as well as a dynamic allocation system.

The predicted patient flow and/or suggested rearrangements of resources can be communicated by wired and/or wireless communication to another device. For example, the system may communicate the patient flow and/or suggested rearrangements to a mobile phone, computer, laptop, wearable device, and/or any other device configured to allow display and/or other communication of the outputted information.

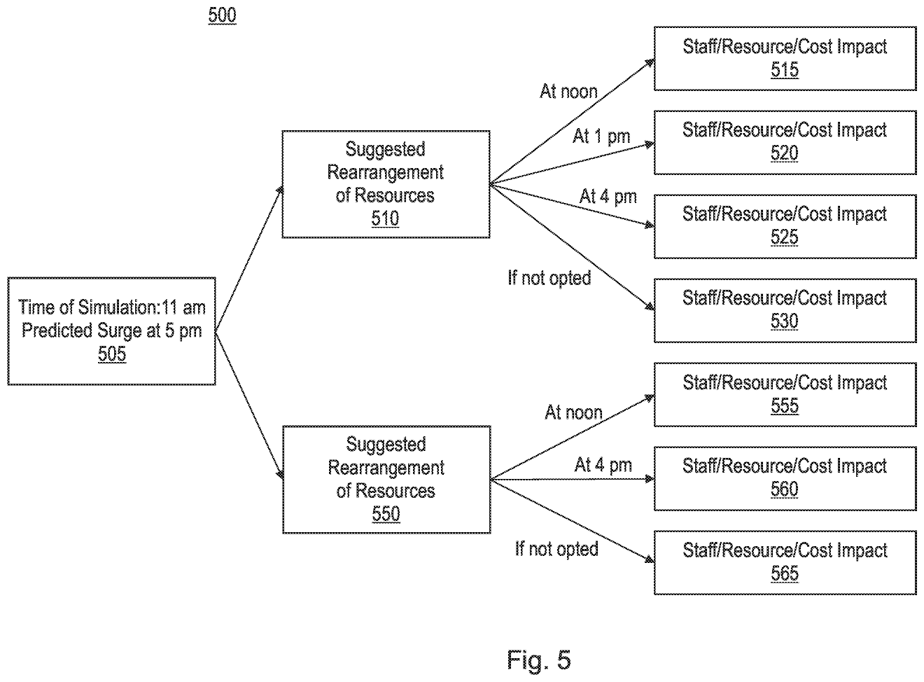

FIG. 5 shows an example schematic graphical representation of a display of a capacity user interface 500. The display can comprise many different configurations and information. For example, the display can comprise the occupancy, capacity, and expected changes in the demand of resources to a hospital. The expected changes can comprise an expected surge in patients, for example. The predicted patient flow, carried out by simulation module 140 or engine 340, can generate a predicted demand or capacity output such as a predicted surge in patients at 5 pm as shown at 505. Such predicted demand or capacity output can represent a potential deviation from the present capacity in the hospital or sub-ward or unit. For example, the present capacity (e.g., a number of beds) can be displayed along with the number of beds that are occupied. An expected number of beds needed for the expected surge can also be displayed. The user can also be presented with at least one or two or more suggested rearrangements of resources to address the predicted surge in patients as shown at 510 and 550. Although FIG. 5 shows only two suggested rearrangements of resources, it should be understood that any number of suggestions can be provided.

The user can simulate each suggestion 510 and 550 using the capacity user interface 500 and visualize the outcome for the next few hours, or for another amount of time up to 72 hours. In example embodiments, the user can visualize the staff, resource, and/or cost impact 515 if the user adopts the suggested rearrangement of resources 510 at 12 noon, an hour from the time of simulation and prediction. The user can additionally or alternatively visualize the staff, resource, and/or cost impact 520 if the user adopts the suggested rearrangement of resources 510 at 1 pm, two hours from the time of simulation and prediction. The user can additionally or alternatively visualize the staff, resources, and/or cost impact 525 if the user adopts the suggested rearrangement of resources 510 at 4 pm, five hours from the time of simulation and prediction. At 530, the user can additionally or alternatively visualize the staff, resources, and/or cost impact if the user opts not to adopt the suggested arrangement of resources 510 at all. The display can be color-coded to highlight those rearrangements that are desirable and/or those that are not in embodiments. For example, routes to 515 and 520 may be desirable and displayed in a first color, whereas the routes to 525 and 530 may not be desirable and displayed in a second color, different than the first color. Of course any number of routes can be desirable or undesirable depending on the scenario.

Similarly, the user can visualize the staff, resource, and/or cost impact 555 if the user adopts the other suggested rearrangement of resources 550 at 12 noon, an hour from the time of simulation and prediction. The user can additionally or alternatively visualize the staff, resource, and/or cost impact 560 if the user adopts the other suggested rearrangement of resources 550 at 4 pm, five hours from the time of simulation and prediction. The user can additionally or alternatively visualize the staff, resource, and/or cost impact 565 if the user opts not to adopt the suggested rearrangement of resources 550 at all. If the suggested rearrangement of resources 510 is preferred over the other suggested rearrangement of resources 550, color coding or any other suitable visual indicia can be used to reflect the preference from the system.

Of course, the staff impacts can be displayed separately from the resource and cost impacts for each route. Additionally, the resource impacts can be displayed separately from the cost impacts for each route. Thus, in embodiments, each impact can be displayed separately so the user can evaluate each individually.

Referring to both FIGS. 4 and 5, at step 440 of the method, the patient flow system receives a selection of a first suggested rearrangement of resources from a user via the interface 500 and the system displays how the selection would affect the hospital in the future. At step 445 of the method, the patient flow system adopts the selection of a first suggested rearrangement of resources and modifies the hospital capacity information based on the adopted selection. In other words, the user can choose to accept or adopt one of the suggestions or recommendations and apply the changes to the database with a single click, for example. In embodiments, the user can simultaneously apply the changes at the hospital or a sub-ward or unit of the hospital. In embodiments, the user's selection can be set for a fixed duration, after which the capacity can be automatically reset to its original configuration or a previous configuration.

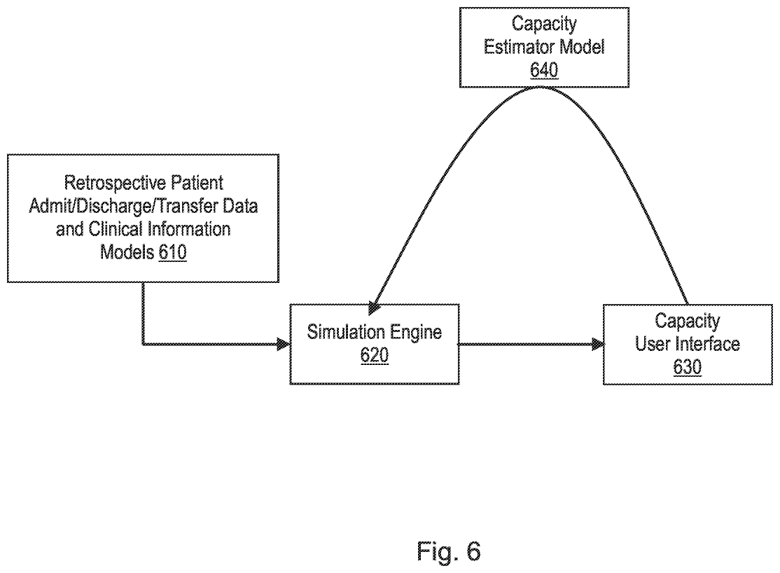

The outcome of the user's selection from the system's suggested rearrangement of resources can be fed back into the simulation at step 450 of the method. An example feedback process is shown in FIG. 6. At 610, output from the retrospective patient admit/discharge/transfer data and clinical information models is fed into simulation engine 620. The prediction from simulation engine 620 is provided to a capacity user interface at 630. Output from a capacity estimator model 640 feeds back into simulation engine 620 based on user input at the capacity user interface 630. The feedback thus can provide a real-time effect on the next iteration of the simulation, thereby ensuring a mirroring of the hospital, programmatically.

The method and system may receive updated information. The updated information may be historical hospital data, hospital capacity information, and/or patient clinical data. For example, the clinical data for the admitted patients may change for the better or the worse which indicates that beds that would have opened up might not be available for new patients. That updated clinical information, such as worsening vital signs or improved prognosis, may be provided to the system. As another example, the hospital bed capacity may change, due to a policy change, and that information may be provided to the system.

With the updated information, the system returns to steps 405, 410, and 420 of the method to update the machine learning algorithm. The system can then generate an updated predicted patient flow, and display the flow and/or any suggested rearrangements of resources. According to an embodiment, the updated information may comprise information about what changed in the information and/or in the suggestion or recommendation for the hospital or a sub-ward or unit.

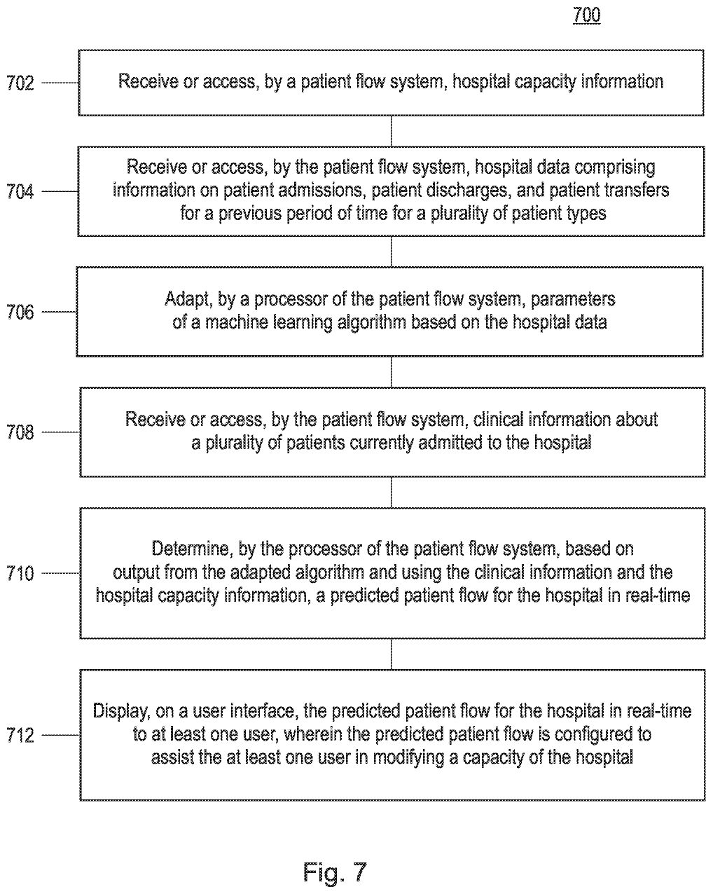

FIG. 7 shows a flowchart of a method for performing a demand analysis using any patient flow system described or otherwise contemplated herein. At step 702 of the method, a patient flow system receives or accesses hospital capacity information. At step 704 of the method, the patient flow system receives or accesses hospital data comprising information on patient admissions, patient discharges, and patient transfers for a previous period of time for a plurality of patient types. At step 706 of the method, a processor of the patient flow system adapts parameters of a machine learning algorithm based on the hospital data. At step 708 of the method, the patient flow system receives or accesses clinical information about a plurality of patients currently admitted to the hospital. At step 710 of the method, the processor determines a predicted patient flow for the hospital in real-time based on output from the adapted machine learning algorithm and using the clinical information and the hospital capacity information. At step 712 of the method, a user interface displays the predicted patient flow for the hospital in real-time to at least one user, wherein the predicted patient flow is configured to assist the at least one user in modifying a capacity of the hospital.

As described herein, the input data comprises patients' trajectory details across a hospital (e.g., unit/ward visited, unit start time, unit end time, encounter class, discharge disposition), admit/final diagnosis and reason for visit data to identify the patient type, and patient vital signs to identify the deterioration level for ward queuing. When the system is deployed, two years of retrospective data can be requested or otherwise retrieved from the hospital IT/data repository to run the adaptive parameter modeling approach. After the deployment of the system, the historical dataset needs to be kept updated with real-time streaming data (HL7/FHIR) to maintain the two year data window. The systems and methods described herein advantageously improve the parameters of the simulation over time to keep the simulation accurate. The parameters are, e.g., patient arrival rates, unit transition probabilities, and length of stay, for example.

By combining historical hospital admission/discharge/transfer data with clinical information in a demand analysis, this novel patient flow system has an enormous positive effect on optimizing patient flow compared to prior art systems. As just one example, the simulation engine can emulate a hospital as a virtual hospital twin, taking into account existing patients in the hospital in real-time and combining with simulated patients to accurately define their trajectories and lengths of stay through the hospital. The simulation engine further predicts utilization of resources like ventilators, medications, and other consumable resources and also the caregiver's time and attention spent on the patient, for which the clinical information aids the algorithms. The high level of detail provided demonstrates the waiting or pooling that can potentially occur when patients transition from one ward or unit to another, where the destination ward or unit may be at capacity.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for performing, using a patient flow system, a demand analysis for a hospital to optimize a flow of patients within the hospital, comprising:
receiving or accessing, by the patient flow system, hospital capacity information about the hospital;
receiving or accessing, by the patient flow system, hospital data about the hospital, wherein the hospital data comprises information on patient admissions, patient discharges, and patient transfers for a previous period of time for each of a plurality of patient types;
adapting, by a processor of the patient flow system, parameters of a machine learning algorithm based on the hospital data;
receiving or accessing, by the patient flow system, clinical information about a plurality of patients currently admitted to the hospital;
determining, by the processor of the patient flow system based on output from the adapted machine learning algorithm and using the clinical information about the plurality of patients currently admitted in the hospital, and the hospital capacity information, a predicted patient flow for the hospital in real-time; and
displaying, on a user interface, the predicted patient flow for the hospital in real-time to at least one user, wherein the predicted patient flow is configured to assist the at least one user in modifying a capacity of the hospital.

2. The method of claim 1, further comprising displaying, on the user interface, to the at least one user a plurality of suggested rearrangements of resources within the hospital based on the generated predicted patient flow.

3. The method of claim 2, further comprising:
receiving a selection, by the at least one user using the user interface, of a first suggested rearrangement of resources of the plurality of suggested rearrangements of resources; and
displaying, on the user interface, how the selected first suggested rearrangement of resources would affect the hospital for at least a few hours in the future from the step of receiving the selection of the first suggested rearrangement of resources.

4. The method of claim 3, further comprising displaying, on the user interface how the selected first suggested rearrangement of resources would affect the hospital for more than 24 hours in the future from the step of receiving the selection of the first suggested rearrangement of resources.

5. The method of claim 2, further comprising:
receiving a selection, by the at least one user using the user interface, a second suggested rearrangement of resources of the plurality of suggested rearrangements of resources; and
displaying, on the user interface, how the selected second suggested rearrangement of resources would affect the hospital for at least a few hours in the future from the step of receiving the selection of the second suggested rearrangement of resources.

6. The method of claim 2, further comprising:
adopting at least one suggested rearrangement of resources of the plurality of suggested rearrangement of resources after receiving user input from the at least one user at the user interface;
modifying the hospital capacity information based on the adopted at least one suggested rearrangement of resources; and
incorporating at least one change to the resources in the hospital.

7. The method of claim 6, further comprising modifying the hospital capacity information for a fixed duration, after which, automatically resetting the hospital capacity information to an original or previous configuration.

8. The method of claim 6, further comprising providing feedback to a simulation engine by inputting the adopted suggested rearrangement into the simulation engine thereby updating a basis for a subsequent simulation.

9. A patient flow system configured to perform a demand analysis for a hospital to optimize a flow of patients within the hospital, comprising:
hospital capacity information about the hospital;
hospital data about the hospital, wherein the hospital data comprises information on patient admissions, patient discharges, and patient transfers for a previous period of time for each of a plurality of patient types;
clinical information about a plurality of patients currently admitted in the hospital;
a machine learning algorithm configured to be adapted based on the hospital data about the hospital;
a processor configured to: (i) adapt parameters of the machine learning algorithm based on the hospital data; (ii) generate a predicted patient flow for the hospital in real-time based on: (1) output from the adapted machine learning algorithm, and (2) the clinical information about the plurality of patients currently admitted in the hospital, and (3) the hospital capacity information; and
a user interface communicably coupled with the processor, the user interface configured to display the predicted patient flow for the hospital in real-time for at least one user, wherein the predicted patient flow is configured to assist the at least one user in modifying a capacity of the hospital.

10. The patient flow system of claim 9, wherein the user interface is further configured to display, to the at least one user, a plurality of suggested rearrangements of resources within the hospital based on the generated predicted patient flow.

11. The patient flow system of claim 10, wherein the user interface is further configured to:
(i) receive user input from the at least one user selecting a first suggested rearrangement of resources of the plurality of suggested rearrangements of resources; and
(ii) display, to the at least one user, how the selected first suggested rearrangement would affect the hospital for at least a few hours in the future after receiving the user input.

12. The patient flow system of claim 10, wherein the user interface is further configured to:
(i) receive user input from the at least one user selecting a second suggested rearrangement of resources of the plurality of suggested rearrangements of resources; and
(ii) display, to the at least one user, how the selected second suggested rearrangement of resources would affect the hospital for at least a few hours in the future after receiving the user input.

13. The patient flow system of claim 10, wherein the processor is further configured to:
(i) adopt at least one suggested rearrangement of resources of the plurality after receiving user input from the at least one user at the user interface; and
(ii) modify the hospital capacity information based on the adopted at least one suggested rearrangement of resources.

14. The patient flow system of claim 13, wherein the hospital capacity information is modified for a fixed duration, after which the hospital capacity information is automatically reset to an original or previous configuration.

15. The patient flow system of claim 13, wherein the processor is further configured to provide feedback to a simulation engine by inputting the adopted suggested rearrangement into the simulation engine thereby updating a basis for a subsequent simulation.
